(19) United States
(12) Patent Application Publication
(10) Pub. No.: US 2022/0211316 A1
(43) Pub. Date: Jul. 7, 2022

(54) Using Reorganization and Plasticity to Accelerate Injury Recovery (URePAIR)

(71) Applicant: Larry Lior Turetsky, Philadelphia, PA (US)

(72) Inventor: Larry Lior Turetsky, Philadelphia, PA (US)

(21) Appl. No.: 17/140,469

(22) Filed: Jan. 4, 2021

(51) Int. Cl. A61B 5/375; A61B 5/38; A61B 5/378; A61B 5/374; A61B 5/00; G09B 19/00

(52) U.S. Cl. CPC A61B 5/375; A61B 5/38; A61B 5/378; A61B 2503/06; A61B 5/6803; G09B 19/00; A61B 5/374

(57) ABSTRACT

The name of this program is: Using Reorganization and Plasticity to Accelerate Injury Recovery (hereafter known as “URePAIR”). This is a request for protection and the exclusive right to my brain injury repair method for the purpose of promoting the progress of behavioral science investigation and clinical interventions to improve mentation and behavior.

### PROFESSIONAL CREDENTIALS

Doctor of Chiropractic, National Board of Chiropractic Examiners academic certification, Post-Doctorate Chiropractic Neurology, Certified Chiropractic Rehabilitation, Diplomate American Academy of Pain Medicine, Diplomate Chiropractic Biophysics, Not licensed to practice nor active member of any association

### SPECIFICATION/BACKGROUND

Today's standard psychological model, that environmental conditions are the cause for mental states and behavior is incorrect and needs to be replaced by a brain based model. This environmental based model has caused a crisis in the clinical outcomes and cultural understanding of the mechanisms that cause mentation and behavior. In the clinical fields, this crisis is most easily seen in the high recidivism rates of addicts and chronic criminals coming from highly funded departments whose expressed mission is to rehabilitate its inmates. The difference between the public's expectation of inmate rehabilitation and actual recidivism rates allows a failed system to continue, in effect, a criminogenic system. Culturally, the crisis is most easily seen in the continued acceptance of religious-based notions of behavior that cause fantastical expectations of redemption, social expectations and political parties/ideology that take the place of rational legislative judgments. Effective solutions remain elusive not from a “bug” in the model, but the model itself is the impediment.

Modern psychology must leave the humanities field and into the fields that make up behavioral neuroscience: physics, chemistry, mathematics and bioengineering. Mental states and behavior are an emergent property of one's connectome (the collection of all neuron synapses and pathways). To the extent some clinicians recognize the brain as the cause of mentation and behavior, their models are too regional or chemical specific, therefore not take account of the dynamic networking of physical connection structures. Whether the subjectivity and vagueness of psychological theory is a product, or a cause of its religious and philosophical roots is irrelevant. Like the medical field, psychology should evolve in a direction of reductionism and materialism. Clinicians must change their fundamental question from “what environmental stimuli should be changed”, to “what change of brain processing can be done”?

The dilution, dismissal or denial of the biological basis of mentation and behavior allows for the accumulation of unnecessary suffering, costs and is resistive to solutions that are possible or already available. It turns out the immaterial products of thoughts, emotions and the behaviors that result are as much a product of a physical structure as walking is a product of our physical skeleton and muscles. Emotions and thoughts are not something we can touch or see, but what creates them can be touched and seen. My hope is that a psychological reform along with a standardized objective model for diagnosis and brain-based treatment program will reduce the suffering and costs that come from outdated and ineffective ideas.

There is an old saying, “when all else fails, read the directions”. Here is how thoughts, emotions and behaviors are caused in all organisms (regardless of their size, weight or complexity) that have at least one neuron. The environment stimulates their sensory system and that information is converted to rapid off/on nerve impulses. Those nerve impulses go directly to and get processed by either a small nerve network of connections or a large network of connections organized as a brain (in humans). This patent is based on the fact that the brain's processing mechanism gets damaged and needs repair prior to, or in conjunction with, environmental based therapy (counseling, education, prison, etc.). If brain processing is unhealthy, then environmental stimuli will be ineffective to improve mentation and behavior long term. This patent is a method to objectify and improve the mechanism that causes of thoughts, emotions and behavior with the goal to reduce human suffering, crime/addiction and to help reach the maximum of one's human potential.

The adult human brain (at 25 years old) is only about 3 pounds and the size of your two fists put together. It contains about 86 billion neurons make about 10¹⁵ (1,000 trillion) connections or synapses. These synapses are connected with about 108,000 miles of nerve extensions, or about 400 feet per cubic millimeter (grain of rice). The synapses and nerve fiber connections naturally reduce with age by about 10% per decade from adulthood, but what remains tend to get stronger and easier to stimulate. The network and connections are not laid out randomly. In fact, it is the unique physical signature connection pattern that gives the brain the processing ability it has. This physical connection network is known as the connectome.

The connectome is the complete description of the structural connectivity (the physical wiring) of an organism's nervous system. Its complex products (thoughts and behaviors) are produced by dividing up the task into many separate and simpler tasks. Like a large company, each smaller task is processed in a unique area designed for that small task. Many small tasks are combined back together for the resultant complex thought or behavior. This process of: digitizing environmental stimuli, dividing, processing, recombining and executing a task (not to mention memorializing and adjustments for learning), happen in less than a second. To repeatedly accomplish the speed and accuracy of such complicated tasks requires a physical connectome structure. Chemical neurotransmitters are vital to the electrical signal propagating through the network, but their relatively small number and chemical similarity can't account the speed and complex tasks that are generated. Large volume changes certainly can explain chronic impairment(s) or disease states. However, the ease of making neurotransmitters from easily diet available amino acids makes the connection stimulating effects on transmitter producing cells the more common problem. You can think of neurotransmitters as the grease that make the gears turn easily, but the physical gears carry the loads and do the work. The field of science dealing with the assembly, mapping and analysis of data on neural connections is called connectomics. The connectome is physically connected in a specific way to produce the unconsciousness and consciousness outputs that allow us to live and experience.

While the connectome certainly receives and is influenced by the environment (parenting, education, vocation, resources, social interactions etc.), the output is based on how the environment is processed. Processing is dependent on the preexisting physical connectome structure. For example, one's dog or cat cannot speak or understand English (simple reflexive actions to conditioned commands is not language) despite having it in its environment. They lack the few genes, that every human baby has, to develop a specialized nerve structure for language comprehension and speech. Neurotransmitters certainly play an important role. However, they only allow an electrical signal to pass through the network. The memories, muscle movements and thoughts that allows our language are stored by physical nerve connections and patterns, not using varied neurotransmitter volumes. The brain does not function as a soup, but functions as an organic computer. A physically damaged or unhealthy connectome structure will not process, memorialize or adapt to the environment properly, therefore the brain's output (thoughts and behavior) will be unhealthy.

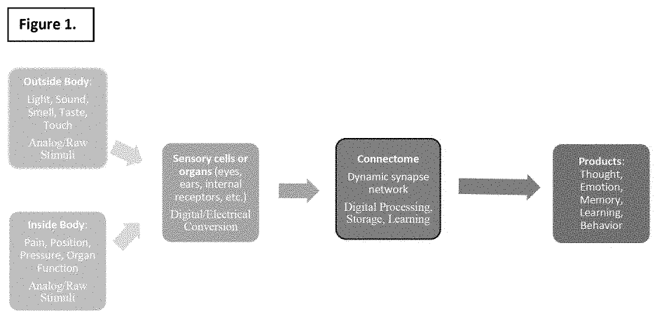

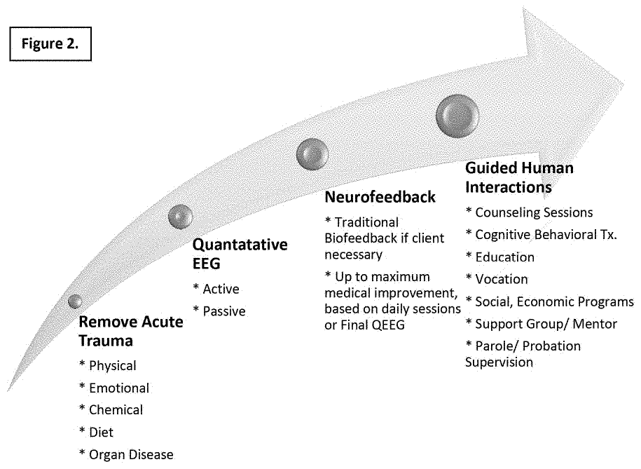

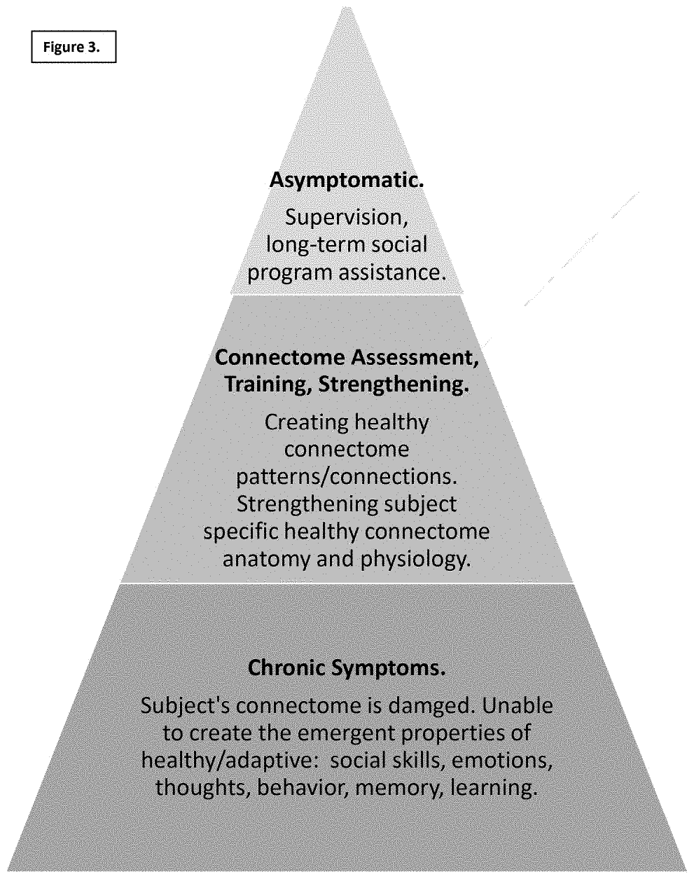

All mental and behavioral phenomena are created (in organisms that have at least one neuron) in two steps. See FIG. 1. First, incoming analog data must be converted into corresponding rapid on/off nerve impulses by the sensory cells and organs. Examples of environmental data are: light, sound, chemicals, temperature, pressure, physical positioning and pressure, etc. You could say that, like a computer keyboard, input is “digitized” into impulses that the processor can interpret. The brain will process information using electrical based synapses and connections, so “outside” analog stimuli must be converted into the language of the brain-electricity. Sensory nerves continuously send the “digital” data to the connectome. This includes data originating from inside or outside the body. Remember, the brain must process inside the body information as well, such as: organ function, movement, pain, etc. All information (except smell) goes to the brain's Thalamus region. The Thalamus distributes the digital signal to various other brain regions for processing. Digitized smell data goes directly, without additional synaptic connection and without the Thalamus relay, to Limbic areas for initial processing and later inclusion with other brain processing. This connectome processing takes several hundred milliseconds.

Connectome creation and modification changes in four general stages. The First is before birth. It is estimated that about half of the total (eventual) connectome pattern is formed prior to birth. The genetic influence is primary, however pre-natal environment is also an influence. For example: mother's diet, stress level, lifestyle factors, fetus physical trauma. The second general stage is post-birth to age seven. During this stage about 95% of connectome is formed. It is in this stage that environment is most influential in connectome formation. A genetic and chemical foundation is designed to form a connectome specific to environmental stressors and adaptive needs, such as: skills learning, language and emotional regulation. For example, the amygdala (area vital to emotional/behavioral regulation) is largely undifferentiated at birth. However, the level of stressors or risks such as danger and violence greatly modify the threshold for amygdala firing into the rest of the connectome. A child with violent parents during this age will have a higher threshold to violence and anger responses. The older child will have more tolerance to giving and receiving violence and anger. In this age range, my protocols are not as effective as the connectome is still developing. Since the environment is primary in connectome development and it is not developed yet, it is best to modify thoughts and behavior using typical conditioning stimuli (school, parenting, positive social feedbacks). The third stage is maturation of the connectome. It occurs from seven to 25 years old. In this stage the existing connectome patterns will be the largest influence on maturation. From this stage on my protocols are most effective. Connectome assessment can be reliable and intervention is easier. Development is still occurring, because brain chemistry is still promotive of synapse creation and strengthening. The fourth stage contains the most network connections and synapse loss. Evolutionary designed brain matter loss is likely because our species did not typically live beyond 25 for most of our existence and a broad set of new skills were not necessary to learn in the “simple” world of hunter gathering or even in the very recent agricultural lifestyle. The brain is designed to prune away connections to make the strongest connections even more relevant to mentation and behavior. Basically, we get better at less diverse skills. It estimated that after 25 we lose about 10% of white matter connections each decade until death. Since willful behavior and conscious control of emotions and thoughts are exclusive to the connectome, and the connectome is overwhelmingly formed without the subject's control, we should view chronic mal-adaptive thoughts and behavior under these constraints. Anti-social behavior should be modified and punished, however we need to remove the stigma associated with diagnosis and treatment and personal judgment from punishment to be effective. This is not a defense or acceptance of maladaptive thoughts and behavior. Quite the opposite. The goal is correction, and having natural vindictive or hatred emotions govern our approach has never been helpful to correction (the goal of everyone). Understanding the brain in these four general stages improves our understanding of environmental stimuli influences and how to correct unhealthy thoughts and behavior.

The output (mentation and behavior) is a direct product of the connectome. If there is an anatomical or physiological abnormality in the connectome, the incoming analog data will not be processed appropriately. For long term improvement of chronic undesirable thoughts or behavior, we should focus on altering the structure and function of what creates the perception, processing and memory out of the environmental stimuli—the connectome.

One can now see three “entry points” were clinical efforts can be placed to reform chronic abnormal thoughts, emotions and behavior. 1) the environment, 2) the sensory cell/organs and 3) the connectome. The environment certainly is an influence of psychological impairment. Poor parenting, traumatic event, poor diet, or medical disease will create unhealthy connection patterns. For several decades there has been study of Adverse Childhood Experiences (ACEs). However, any type of trauma that causes a chronic psychological impairment must have, by definition, been done to the anatomical and physiological connectome. Where else would any memory or location of processing be? Since the trauma is in the past, the focus must be to the damaged connectome. Obviously, if there is an ongoing medical disease or ongoing physical or emotional trauma, those must be removed from the potential experience of the subject before connectome repair. This treatment model is like any other medical problem, for example a heart attack. Years of smoking, poor diet and lack of exercise can cause arteriosclerosis. This physical arterial damage can lead to heart failure during a large physical exertion. The medical standard of care would be to (in order): remove the exertion, return heart and lungs to normal rhythm/rate, remove the physical arteriosclerosis, and finally, lifestyle education (diet, smoking, stress and exercise counseling). Counseling before the accumulated physical damage is treated would not only be less effective but could worsen a damaged cardiovascular system. The central nervous system is no different.

The second “entry point” is the sensory cells/organs. If there is blindness from loss of an eye, deafness from inner ear damage or chronic pain from hypersensitivity (i.e. an amputation), those issues will affect what the connectome can process, but not a job for psychologists or clinical neuroscience. These will need medical care. The instant process can still be exercised with recognition of sensory system limits.

The name of this program model is Using Reorganization and Plasticity to Accelerate Injury Recovery model (hereafter known as “URePAIR”). The goal of URePAIR is to evaluate neural computation for clinical use, reorganize brain cell architecture and enhance natural brain plasticity (specific to subject's needs) for long-term improvement in mental states and behavior. URePAIR will create an expanded neuroscience community, bringing together previously separate but complementary approaches to solve old problems and address new ones. The program will also improve our ability to treat and restore the injured brain through development of neural interfaces and adaptive strategies derived from 21^{st} century brain models. While this method is most needed at this time in addiction/substance use disorders and chronic criminal behavior, said method can and should be used, also by qualified professionals, for other mental state and behavioral problems.

This Utility Patent application focuses on a method to improve brain function of an individual in order to improve their success with daily hying, educational, vocational and social activities. The idea being that healthy brain function will allow for improved skills training in the above activities. Healthy brain function will be measured using a Quantitative Electroencephalograph, commonly called a QEEG or functional EEG brain map. QEEG can be active or passively performed. Active QEEG uses specific stimuli or test protocols during the gathering of brain wave data, such as: eyes open/closed, reading/memory/spatial-based questions. Passive QEEG does not use specific subject stimuli. Subject's brain wave measurements are taken during simple relaxed positioning. Improvement in the function of the brain is accomplished using specifically tailored (based on the QEEG) Neurofeedback based training. Neurofeedback training is also commonly called EEG-Biofeedback or Neuromodulation. The goal of Neurofeedback is to “normalize” measured brain wave patterns of a subject. Normalize can mean to increase or decrease a single, or several brain waves, amplitude in a given brain location. Initial brain wave measurements are obtained on the initial QEEG test. Monitoring of clinically relevant frequency/location occurs throughout the neurofeedback treatment sessions. Normal threshold amplitude and location is defined by studies of asymptomatic individuals of a specific sex, age and other cohort factors. Human guided skills training is also used to improve said activities. Experienced/qualified humans will interact with a subject in typical settings with the goal of strengthening and increasing the distribution of neural connection patterns. Examples of human guided training are: counselors, psychologists, social workers, teachers, vocational training, etc. Their interaction with a subject will stimulate a brain to create connections in a pattern consistent with the stimuli (i.e. talk, classrooms, real time training). Dependent on the needs of the individual, human based skills training can occur prior to, during and or after Neurofeedback training. Essentially, a better functioning brain can better perceive, incorporate, memorialize and utilize daily living, educational and social skills training.

Nature has unchanging rules. We can use this to our advantage if we learn and use the rules. Regarding thoughts and behavior, rule number one is that the brain is the cause. Rule number two is that the structure of the network connections dictates its function (actually, the structure of anything dictates its function). Each connectome has a unique structure that constrains the dynamic processing of, environmental stimuli and causes our personality, thoughts, emotions and behavior. Chronic poor judgments and behaviors (hurting ourselves and/or others) are caused by a damaged connectome. I propose a neurobiological method to improve neural architecture in order to improve mental states and behavior. My method uses brain waves as the agent to measure and improve connectome function. A clinical intervention uses computer-based interaction to re-condition the dynamic connectome function. Such brain wave condition training helps set up the brain's adaptive mechanisms to better incorporate incoming environmental stimuli (sound, visual, olfactory and other sensory input). Improvement in connectome function allows for more effective and further development of neural plasticity via guided human interactions (counseling, education, behavioral modification techniques, etc.). This model is different from other patents because of the following factors, as a whole and/or in part: 1) it approaches mental state and behavior analysis and modification by continuously incorporating subject's EEG brain waves; 2) pre-treatment analysis of subject's neurological state uses a standard 10-20 system setup; 3) it uses EEG training computers combining Analog/Digital converters with Fast Fourier Transformers; 4) continuously uses subject's EEG waves to feedback into visual and audio stimulation designed to condition new brain wave patterns; 5) analyzes near real-time EEG waves while modifying mental states and behavior by altering neuronal architecture/circuits; 6) it combines brain wave training followed by specific and guided human interaction; 7) for addictive disorders, it requires an inpatient detoxification phase; 8) for all mental and behavioral disorders, it requires a supportive care phase of treatment; 9) measures and trains subject specific EEG frequencies by using absolute powers and relative powers in specific brain locations; 10) it is not invasive nor uses pharmaceuticals; 11) the required tools and methods are currently within the financial scope and easily accessible to independent clinicians.

Our success, as individuals or a species, is not a natural consequence to be expected. Nor has history shown progress to be a series of gradual rationally based resolutions of philosophical consequences. “Mother nature” can equally accept humans as masters of our domain or fools causing our own suffering or extinction. Despite everyone's right to freedom of expression and belief, we cannot continue to teach or accept outdated and ineffective psychological models. If we are to succeed with mental health issues that overwhelm our chronic: homeless, criminals, addicts, impoverished, and DSM-5 diagnosable, we will need to adopt neuroscience findings-even if those findings do not agree with our already held beliefs and teachings on the subject. Harvard Professor of Psychology and best-selling author, Steven Pinker, said, “The refusal to acknowledge human nature is like the Victorians' embarrassment about sex, only worse: it distorts our science and scholarship, our public discourse, and our day-to-day lives. Logicians tell us that a single contradiction can corrupt a set of statements and allow falsehoods to proliferate through it. The dogma that human nature does not exist, in the face of evidence from science and common sense that it does, is just such a corrupting influence.” [Pinker, Steven, The Blank Slate: The Modern Denial of Human Nature. Penguin Books, preface, page ix, 2002]

1. An active electrophysiological assessment of a child (≥eight years old) or adult subject using a Quantitative Electroencephalograph (QEEG), also commonly known as brain mapping. Auditory and visual stimuli are presented to a subject, via a computer interface and traditional EEG scalp/head connections. An EEG electro-cap is placed on a subject's head to measure QEEG brain variables in 19 locations (International 10-20 system) comprising of coherence, phase, relative power, peak frequency, peak amplitudes, and magnitudes at 5 frequency bands comprised of 0-4 Hz (Delta), 4-8 Hz (Theta), 8-13 Hz (Alpha), 13-32 Hz (Beta), & 32-64 Hz (High Beta/Gamma). During said measurement parameters, the subject is engaged in specific repetitive reading, auditory memory and problem-solving tasks. The subject's EEG values, during the tasks are monitored and recorded via examiner's own computer interface. The subject's EEG data following the tasks are compared, using computer analysis, to a database that matches the subject's age, gender and clinically symptomatic cohort. The data is analyzed for deviation from the average values on the variables which are related (positively and negatively) to performance in the relevant database. The subject's analysis in phase and coherence variables in at least the 13-32 & 32-64 Hz bands for deviation from the average values of the 19 QEEG variables which are positively or negatively related to performance in the relevant database. The subject's raw QEEG data and said comparative analysis results are recorded. In the case of addiction or substance abuse subjects, active QEEG is performed after a medically supervised detoxification program is successfully completed.

2. A passive electrophysiological assessment of a child (≥eight years old) or adult subject using a Quantitative Electroencephalograph (QEEG), also commonly known as brain mapping. An EEG electro-cap is placed on a subject's head, while seated upright, to measure QEEG brain variables in 19 locations (International 10-20 system) comprising of coherence, phase, relative power, peak frequency, peak amplitudes, and magnitudes at 5 frequency bands comprised of 0-4 Hz (Delta), 4-8 Hz (Theta), 8-13 Hz (Alpha), 13-32 Hz (Beta), & 32-64 Hz (High Beta/Gamma). The subject's EEG values, during the tasks are monitored and recorded via examiner's own computer interface. The subject's EEG data following the tasks are compared, using computer analysis, to a database that matches the subject's age, gender and clinically symptomatic cohort. The data is analyzed for deviation from the average values on the variables which are related (positively and negatively) to performance in the relevant database. The subject's analysis in phase and coherence variables for deviation from the average values of the 19 QEEG variables which are positively or negatively related to performance in the relevant database. The subject's raw QEEG data and said comparative analysis results are recorded. In the case of addiction or substance abuse subjects, passive QEEG is performed after a medically supervised detoxification program is successfully completed.

3. Brain based electrophysiological training sessions. Neurofeedback (NF), or also commonly known as EEG Biofeedback or neuromodulation, is a modality used to increase or decrease one or more of the subject's deviated (relative to above QEEG analysis and asymptomatic similar cohorts) EEG variables. An electro-cap is placed on the subject's head for standard EEG electrode 10-20 placement. A NF device provides the auditory and/or visual stimuli via subject's computer interface. An examiner, via his/her own connected computer interface, monitors, records and adjusts the relevant EEG training variables. The NF training session strategy is to increase and/or decrease one or more of the positively related QEEG variables until the value of the one or more QEEG variable is at the average value or above the relevant database. The NF session(s) goal is to “normalize” EEG variations found during the reading, memory and problem-solving tasks compared to a similar cohort. Training occurs during each session and done in a method to gradually “normalize” the deviant EEG variable(s) by using progressively increasing the difficulty to meet the EEG variable training thresholds. The purpose real-time gradual and progressive threshold management is allow natural brain plasticity to create and strengthen neuron connections and network pathways to make long-term improvement and stability. The subject's NF session raw and performance data are monitored and recorded for use during each session. Sessions are performed once per day for multiple times per week (generally two to three/week) until subject reaches maximal medical improvement (generally ≤40 sessions per condition). Session frequency and gross total are calibrated to patient compliance ability and progress outcomes. In a minority of subjects, NF can be too difficult to begin with for brain wave conditioning/learning. In such cases, traditional biofeedback must be done prior to NF. Traditional biofeedback does not use EEG brain wave to during training sessions, but rather trains to alter breathing rate, heart rate, muscle contraction and sweat/temperature. The expectation is as EEG variable deviancy is lessened, thoughts, emotions and/or behavior will correspondingly improve. Because said mentation and behavior are known to be an emergent property of brain function and brain function is a direct product of brain wave production, improvement in mentation and behavior can be associated with improvement in brain wave function.

4. Human guided interactions are done during or after NF program completion. The subject interacts with experienced and qualified humans to learn and perform daily living, educational, vocational and/or social activities. With a healthy or improved connectome, following NF, HGI training is used to increase new nerve connections, strengthen existing nerve connections and increase nerve connection distribution into other associated nerve network areas used for a particular task. Examples of HGI are: individual or group counseling, cognitive behavioral therapy, secular education, vocation training, social skill training and social programs to support or monitor the subject (probation/parole, social worker, mentor). While HGI is already the standard of care for mental or behavioral symptoms, using QEEG and/or NF before or in conjunction with HGI is not.
